(19) United States
(12) Patent Application Publication
(10) Pub. No.: US 2009/0048317 A1
(43) Pub. Date: Feb. 19, 2009

(54) Formulations of candesartan

(76) Inventors: Michael Fox, Tel Aviv (IL); Minutza Leibovici, Netanya (IL); Itamar Kanari, Kefar Sava (IL)

Correspondence Address: KENYON & KENYON LLP, ONE BROADWAY, NEW YORK, NY, 10004, US

(21) Appl. No.: 12/221,365

(22) Filed: Aug. 1, 2008

Related U.S. Application Data
(60) Provisional application No. 60/963,014, filed on Aug. 1, 2007.

(51) Int. Cl. A61K 31/41; A61P 9/00

(52) U.S. Cl. 514/381

(57) ABSTRACT

The present invention encompasses pharmaceutical compositions comprising candesartan and processes for preparing the same. In particular, a pharmaceutical composition comprising candesartan or a prodrug thereof or an analog thereof or a derivative thereof and at least one non-ionic surfactant, wherein the pharmaceutical composition has about 0.01% to about 10% of at least one non-ionic surfactant present in the total weight of the composition.

### CROSS-REFERENCE TO RELATED APPLICATIONS

This application claims the benefit of U.S. provisional application Ser. No. 60/963,014, filed Aug. 1, 2007, hereby incorporated by reference.

### FIELD OF INVENTION

The present invention encompasses pharmaceutical compositions comprising candesartan and processes for preparing the same. In particular, the pharmaceutical compositions comprise candesartan cilexetil.

### BACKGROUND OF THE INVENTION

Drug formulation (immediate release, excipients used, manufacturing methods, modified release—delayed release, extended release, sustained release, etc.) can affect the bioavailability of a drug. In particular, the bioavailability of a drug can be limited by poor dissolution of the drug after being administrated non-intravenously.

Drugs with low aqueous solubility often exhibit poor dissolution rates. Examples of drugs with low aqueous solubility include, but are not limited to, albuterol, zolpidem tartrate, omeprazole, paclitaxel, quetiapine fumarate, alprostadil, candesartan, risperidone, carvedilol, celecoxib, ciprofloxacin, or alprazolam. These drugs with low aqueous solubility often need to be formulated in a manner that increases drug solubility and thus bioavailability.

Candesartan (CNS) is a potent, long-acting, selective AT₁ subtype angiotensin II receptor antagonist. Candesartan is a useful therapeutic agent for treating circulatory system diseases such as hypertensive diseases, heart diseases (e.g. hypercardia, heart failure, cardiac infarction, etc.), strokes, cerebral apoplexy, and nephritis, among others. Candesartan meets the requirement of high potency but it is poorly absorbed when administered orally. Therefore, the prodrug candesartan cilexetil was developed. During absorption from the gastrointestinal tract candesartan cilexetil is rapidly and completely hydrolyzed to candesartan. The chemical name for candesartan is: 2-ethoxy-1-[[2′-(1H-tetrazol-5-yl)biphenyl-4-yl]methyl]-1H-benzimidazole-7-carboxylic acid. The chemical name for candesartan cilexetil is (±)-1-[[(cyclohexyloxy)carbonyl]oxy]ethyl-2-ethoxy-1-[[′-(1H-tetrazol-5-yl)[1,1-biphenyl]-4-yl]methyl]- 1H-benzimidazole-7-carboxylate.

Candesartan cilexetil is a white to off-white powder and is sparingly soluble in water and in methanol. Although candesartan cilexetil contains an asymmetric center in the ester portion of the molecule, it is sold as the racemic mixture.

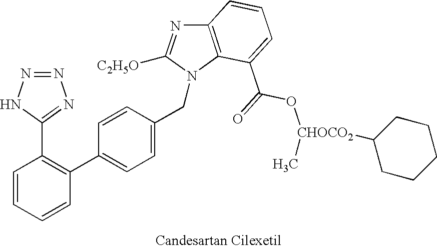

Angiotensin II is formed from angiotensin I in a reaction catalyzed by angiotensin-converting enzyme (ACE, kininase II). Angiotensin II is the principal pressor agent of the renin-angiotensin system, with effects that include vasoconstriction, stimulation of synthesis and release of aldosterone, cardiac stimulation, and renal reabsorption of sodium. Angiotensin II help maintain constant blood pressure despite fluctuations in a person's state of hydration, sodium intake and other physiological variables. Angiotensin II also performs the regulatory tasks of inhibiting excretion of sodium by the kidneys, inhibiting norephedrine reuptake and stimulating aldosterone biosynthesis. Candesartan blocks the vasoconstrictor and aldosterone secreting effects of angiotensin II by selectively blocking the binding of angiotensin II to the AT₁ receptor in many tissues, such as vascular smooth muscle and the adrenal gland. By inhibiting angiotensin II binding to AT₁ receptors, candesartan disrupts the vasoconstriction mediated by AT₁ receptors. Blocking vasoconstriction by angiotensin II has been found to be beneficial to patients with hypertension. The United States Food and Drug Administration has approved candesartan for the treatment of hypertension alone or in combination with other antihypertensive agents. Candesartan cilexetil is marketed in the United States under the trade name Atacand®. Atacand® tablets contain candesartan cilexetil and the following excipients: hydroxypropyl cellulose, polyethylene glycol, lactose, corn starch, carboxymethylcellulose calcium, and magnesium stearate.

European Patent Application No. EP0459136A describes candesartan cilexetil, a process for the preparation thereof, and capsules/tablets comprising the same.

European Patent Application No. EP0546358A describes pharmaceutical compositions comprising inter alia candesartan cilexetil and an oily substance having a melting point of 20 to 90° C.

There have been many efforts directed at enhancing the rate of dissolution of the drug, for example, enhancing dissolution rates by mixing a poorly soluble drug powder with a water-soluble gelatin, which supposedly makes the surface of the drug hydrophilic (Imai, et al., J. Pharm. Pharmacol, 42:615-19 (1990)).

U.S. Pat. No. 6,932,983 refers to porous drug matrices and methods of manufacture thereof, which enhance dissolution of the drug in aqueous media.

Efforts have also been directed at improving the bioavailability of candesartan and cadesartan cilexetil compositions.

US 2008/0058399 refers to the use of solubilizers in order to try to improve bioavailability. However, production parameters that are not commercially industrially applicable are used. For example, micronization, solid solution formation, use of high temperatures, and the use of large amounts of solubilizer. Further, the use of high temperatures may possibly lead to a loss of stability of the product.

In view of the foregoing, there is a need to develop a composition with improved bioavailability. There is also a need to develop a composition with improved bioavailability in which composition stability is not compromised.

### SUMMARY OF THE INVENTION

In one embodiment, the present invention encompasses a pharmaceutical composition comprising candesartan or a prodrug, an analog or a derivative thereof and about 0.01 to about 10% by weight of the pharmaceutical composition of at least one non-ionic surfactant.

Preferably, the prodrug, analog or derivative of candesartan is candesartan cilexetil.

Optionally the pharmaceutical composition further comprises one or more further pharmaceutically acceptable excipients.

In another embodiment, the present invention includes a process for preparing a pharmaceutical composition comprising candesartan or a prodrug, an analog or a derivative thereof and about 0.01 to about 10% by weight of the pharmaceutical composition of at least one non-ionic surfactant wherein the process comprises granulating candesartan or a prodrug, an analog or a derivative thereof, about 0.01 to about 10% by weight of the pharmaceutical composition of at least one non-ionic surfactant and optionally one or more further pharmaceutical acceptable excipients. Preferably, the granules are wet-granulated.

In yet another embodiment, the present invention presents a pharmaceutical composition comprising candesartan or a prodrug, an analog or a derivative thereof and about 0.01 to about 10% by weight of the pharmaceutical composition of at least one non-ionic surfactant obtainable by the process defined above.

In another embodiment, the present invention presents a granule comprising candesartan or a prodrug, an analog or a derivative thereof, about 0.01 to about 10% by weight of the pharmaceutical composition of at least one non-ionic surfactant and optionally one or more pharmaceutically acceptable excipients. Preferably, the granule is wet-granulated.

In another embodiment, the present invention encompasses a pharmaceutical composition comprising a granule as defined above.

In yet another embodiment, the present invention presents a use of a granule as defined above in the preparation of a pharmaceutical composition. Preferably, the pharmaceutical composition is in the form of a tablet.

In a further embodiment, the present invention comprises a method of treating a patient suffering from a disease comprising administering to a patient in need thereof a therapeutically effective amount of a pharmaceutical composition of the present invention. Preferably, the disease is a circulatory system disease such as hypertension.

In a further embodiment, the present invention comprises a pharmaceutical composition of the invention for use as a medicament, preferably for use in treating and/or preventing a circulatory system disease such as hypertension.

### DETAILED DESCRIPTION OF THE INVENTION

The present invention encompasses pharmaceutical compositions comprising candesartan or a prodrug, an analog or a derivative thereof and about 0.01 to about 10% by weight of the pharmaceutical composition of at least one non-ionic surfactant. Preferably, the pharmaceutical composition of the present invention comprises granulates and is preferably compressed granulates. Preferably, the granulates comprise candesartan.

The pharmaceutical compositions of the present invention may be prepared by wet or dry granulation and direct compression. Preferably, the pharmaceutical compositions are prepared by wet or dry granulation, preferably wet granulation. Preferably, the prodrug, analog or derivative of candesartan is candesartan cilexetil.

Candesartan cilexetil can be prepared using any method known in the art. For example, according to US Publication No. 2005/0250827, substantially pure candesartan cilexetil is prepared by providing cilexetil trityl candesartan, deprotecting cilexetil trityl candesartan in a mixture of water and methanol to obtain a residue of candesartan cilexetil; crystallizing the residue of candesartan cilexetil using methanol and toluene; and recrystallizing the crystalline candesartan cilexetil in methanol to yield a substantially pure candesartan cilexetil.

Typically, at least one non-ionic surfactant is selected from the group consisting of block-copolymers of polyoxypropylene (poly(propylene oxide)) and polyoxyethylene (poly(ethylene oxide)) and hydrogenated triglyceride esters of ricinoleic, oleic, and linoleic acids or combinations thereof. Preferably one or two non-ionic surfactants are included in the pharmaceutical compositions of the present invention, more preferably, one non-ionic surfactant is used. Preferably, the non-ionic surfactant is a poloxamer or hydrogenated castor oil powder or a combination thereof. More preferably, the non-ionic surfactant is a poloxamer, most preferably, poloxamer 407 or similar.

As stated above, preferably the non-ionic surfactant component is a poloxamer. A poloxamer is a block copolymer of ethylene oxide and propylene oxide. Poloxamers are also often referred to as polyethylene-propylene glycol copolymers or polyoxyethylene-polyoxypropylene copolymers. Available poloxamers include Lutrol® F127 (Poloxaner 407, BASF).

The amount of non-ionic surfactant present in a pharmaceutical composition is about 0.01 to about 10% by weight of the pharmaceutical composition. Preferably, it is present in an amount of about 0.01 to about 8% by weight of the pharmaceutical composition and more preferably, about 0.01 to about 5%, yet more preferably about 0.01 to about 2.2%, most preferably about 0.01 to about 2.0% by weight of the pharmaceutical composition.

The amount of non-ionic surfactant present per dosage form unit may be between about 0.5 to about 20 mg, preferably about 0.5 to about 15 mg, for example, about 0.5 to about 8 mg, more preferably about 2 to about 10 mg, for example about 2 to about 6 mg or about 2.5 to about 5.5 mg, yet more preferably about 5 to about 8 mg.

The amount of candesartan or a prodrug, an analog or a derivative thereof present per dosage form unit may be between about 2 mg and about 40 mg, preferably about 10 mg to about 35 mg. For example, a pharmaceutical composition in the form of a tablet may contain 2, 4, 8, 16, 32 mg of candesartan or a prodrug, an analog or a derivative thereof.

The pharmaceutical compositions of the present invention may further contain one or more additional excipients including, but not limited to, fillers, diluents, disintegrants, glidants, lubricants, carriers, bulking agents, binders, wetting agents, flavoring agents and the like.

Suitable fillers and diluents include, but are not limited to, cellulose-derived materials like powdered cellulose, microcrystalline cellulose (e.g. Avicel®), microfine cellulose, methyl cellulose, ethyl cellulose, hydroxyethyl cellulose, hydroxypropyl cellulose (e.g. Klucel®), hydroxypropyl methylcellulose, carboxymethyl cellulose salts (such as carboxymethyl cellulose calcium) and other substituted and unsubstituted celluloses; starch such as maize starch; pregelatinized starch; lactose, preferably lactose monohydrate (e.g. Pharmatose®); talc; waxes; sugars; sugar alcohols like mannitol and sorbitol; acrylate polymers and copolymers; dextrates; dextrin; dextrose; maltodextrin; pectin; gelatin; inorganic diluents like calcium carbonate, dibasic calcium phosphate dihydrate, tribasic calcium phosphate, calcium sulfate, magnesium carbonate, magnesium oxide, sodium chloride and other diluents known to the pharmaceutical industry.

Most preferred fillers include lactose monohydrate, pregelatinized starch, mannitol or sorbitol. When sugars are used as fillers, the amount of sugar present in a pharmaceutical composition is at about 30%-80% and preferably about 40%-60% by weight of the pharmaceutical composition. When a starch is used as a filler, the amount of starch present in a pharmaceutical composition is about 5%-50% and preferably about 8%-15% by weight of the pharmaceutical composition.

Suitable disintegrants include croscarmellose sodium (e.g. Ac Di Sol®, Primellose®), crospovidone (e.g. Kollidon(®, Polyplasdone®), microcrystalline cellulose, polacrilin potassium, powdered cellulose, pregelatinized starch, sodium starch glycolate (e.g. Explotab®, Primoljel®) and starch.

Glidants can be added to improve the flowability of a solid composition before compaction and to improve the accuracy of dosing especially during compaction and capsule filling. Excipients that may function as glidants include colloidal silicon dioxide, magnesium trisilicate, powdered cellulose, and talc.

A lubricant may be added to the pharmaceutical compositions of the present invention to reduce adhesion and/or ease the release of the product from e.g. the die. Suitable lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium lauryl sulfate, sodium stearyl fumarate, stearic acid, talc and zinc stearate. Magnesium stearate is most preferred.

Carriers include, but are not limited to, lactose, white sugar, sodium chloride, glucose, urea, starch, calcium carbonate, kaolin, crystalline cellulose, and silicic acid.

Binders include, but are not limited to, carboxymethyl cellulose, shelac, methyl cellulose, potassium phosphate, and polyvinylpyrrolidone. Other suitable binders include, but are not limited to, acacia gum, pregelatinized starch, sodium alginate, glucose and other binders used in wet and dry granulation and direct compression tableting processes.

Flavoring agents and flavor enhancers make the dosage form more palatable to the patient. Common flavoring agents and flavor enhancers for pharmaceutical products that can be included in the composition of the present invention include, but are not limited to, maltol, vanillin, ethyl vanillin, menthol, citric acid, fumaric acid, ethyl maltol, and tartaric acid.

Other excipients that may be incorporated into the formulation include preservatives, surfactants, antioxidants, colourings (e.g. iron oxide red) or any other excipient commonly used in the pharmaceutical industry. Preferably, the pharmaceutical composition may include additional excipients such as, croscarmellose sodium, iron oxide red, spray dried lactose, starch, hydroxypropyl cellulose, sodium lauryl sulfate, microcrystalline cellulose and magnesium stearate.

The pharmaceutical composition may take any form but is preferably a solid composition. More preferably, the pharmaceutical composition of the invention is a compressed solid composition. Suitable solid dosage forms include, but are not limited to, tablets, capsules, powders, or sachets.

Many tablets today are coated after being pressed. A coating may be applied to hide the taste of the tablet's components, to make the tablet smoother and easier to swallow, and to make it more resistant to the environment, extending its shelf life. Tablets may be coated with a variety of commonly known coating materials, for example, tablets may be coated with sugar, gelatin film, or enteric coating. There are also double layered tablets and multi-layered tablets.

When tablets and powders are coated with an enteric coating. The enteric coated powder forms may have coatings including, but not limited to, phthalic acid cellulose acetate, hydroxypropylmethyl-cellulose phthalate, polyvinyl alcohol phthalate, carboxymethylethylcellulose, a copolymer of styrene and maleic acid, a copolymer of methacrylic acid and methyl methacrylate, and the like, and if desired, they may be employed with suitable plasticizers and/or extending agents.

When the pharmaceutical composition is in the dosage form of a capsule, the capsule may contain the pharmaceutical composition of the invention in a form of uncompressed or compressed granulates or powder mixes, etc. The capsules may be covered with either a hard shell or a soft shell. The shells may be made from, but not limited to gelatin and optionally contain a plasticizer such as glycerin and sorbitol, and an opacifying agent or colorant.

Methods of administration of a pharmaceutical composition for treating circulatory system diseases of the present invention are not specifically restricted, and can be administered in various preparations depending on the age, sex, and symptoms of the patient. Suitable routes for administrating a pharmaceutical composition may include, but not limited to, oral, buccal, and rectal administration. Although the most suitable administration in any given case will depend on the nature and severity of the condition being treated, the most preferred route of administration of the present invention is oral.

The amount of candesartan cilexetil contained in a pharmaceutical composition for treating circulatory system diseases according to the present invention is not specifically restricted, however, the dose should be sufficient to treat, ameliorate, or reduce the symptoms associated with the circulatory system disease. The dosage of a pharmaceutical composition for treating circulatory system diseases according to the present invention will depend on the method of use, the age, sex, and condition of the patient. Preferably, about 2 mg to 32 mg of candesartan cilexetil may be contained in a dosage form unit.

In one embodiment, the present invention encompasses a process of preparing a pharmaceutical composition as described above comprising combining candesartan or a prodrug, an analog or a derivative thereof and about 0.01 to about 10% by weight of the pharmaceutical composition of at least one non-ionic surfactant and optionally one or more further pharmaceutically acceptable excipients.

Preferably, the prodrug, analog or derivative of candesartan is candesartan cilexetil.

The amount of non-ionic surfactant present in a pharmaceutical composition is about 0.01 to about 10% by weight of the pharmaceutical composition. Preferably, it is present in an amount of about 0.01 to about 8% by weight of the pharmaceutical composition and more preferably, about 0.01 to about 5%, yet more preferably about 0.3 to about 2.2%, most preferably about 0.5 to about 2.0% by weight of the pharmaceutical composition.

The amount of non-ionic surfactant present per dosage form unit may be between about 0.5 to about 20 mg, preferably about 0.5 to about 15 mg, for example, about 0.5 to about 8 mg, more preferably about 2 to about 10 mg, for example about 2 to about 6 mg or about 2.5 to about 5.5 mg, yet more preferably about 5 to about 8 mg.

The amount of candesartan or a prodrug, an analog or a derivative thereof present per dosage form unit may be between about 2 mg and about 40 mg, preferably about 10 mg to about 35 mg. For example, a pharmaceutical composition in the form of a tablet may contain 2, 4, 8, 16, 32 mg of candesartan or a prodrug, an analog or a derivative thereof.

In another embodiment, the present invention includes a process for preparing a pharmaceutical composition as described above comprising candesartan or a prodrug, an analog or a derivative thereof and about 0.01 to about 10% by weight of the pharmaceutical composition of at least one non-ionic surfactant wherein the process comprises granulating candesartan or a prodrug, an analog or a derivative thereof, about 0.01 to about 10% by weight of the pharmaceutical composition of at least one non-ionic surfactant and optionally one or more further pharmaceutical acceptable excipients. The granulation step may be a wet-granulation or a dry-granulation. Preferably, the granules are wet-granulated.

Preferably, the prodrug, analog or derivative of candesartan is candesartan cilexetil.

The amount of non-ionic surfactant present in a pharmaceutical composition is about 0.01 to about 10% by weight of the pharmaceutical composition. Preferably, it is present in an amount of about 0.01 to about 8% by weight of the pharmaceutical composition and more preferably, about 0.01 to about 5%, yet more preferably about 0.3 to about 2.2%, most preferably about 0.5 to about 2.0% by weight of the pharmaceutical composition.

The amount of non-ionic surfactant present per dosage form unit may be between about 0.5 to about 20 mg, preferably about 0.5 to about 15 mg, for example, about 0.5 to about 8 mg, more preferably about 2 to about 10 mg, for example about 2 to about 6 mg or about 2.5 to about 5.5 mg, yet more preferably about 5 to about 8 mg.

The amount of candesartan or a prodrug, an analog or a derivative thereof present per dosage form unit may be between about 2 mg and about 40 mg, preferably about 10 mg to about 35 mg. For example, a pharmaceutical composition in the form of a tablet may contain 2, 4, 8, 16, 32 mg of candesartan or a prodrug, an analog or a derivative thereof.

Additional excipients such as those described above may be combined with the candesartan or a prodrug, an analog or a derivative thereof and the at least one non-ionic surfactant. The excipients may be added at the same time as candesartan or a prodrug, an analog or a derivative thereof and the non-ionic surfactant are combined. Optionally, additional excipients may be introduced after candesartan or a prodrug, an analog or a derivative thereof and the non-ionic surfactant are combined.

The process of the invention may comprise at least one compression step. Preferably, the compression step is a direct compression process. In a direct compression process, the process of the invention comprises combining candesartan or a prodrug, an analog or a derivative thereof, about 0.01 to about 10% by weight of the pharmaceutical composition of at least one non-ionic surfactant and optionally one or more further pharmaceutical acceptable excipients, and compressing the resultant combination into a solid pharmaceutical composition, preferably a tablet.

In a further embodiment, the compression step includes a mixture made by dry granulation or direct compression.

In one embodiment, the present invention encompasses the use of at least one non-ionic surfactant in increasing the bioavailability of candesartan or a prodrug thereof or an analog thereof or a derivative thereof in a pharmaceutical composition comprising candesartan or a prodrug thereof or an analog thereof or a derivative thereof and said at least one non-ionic surfactant, wherein said at least one non-ionic surfactant is present at about 0.01% to about 10% by weight of the pharmaceutical composition.

Having described the invention with reference to certain preferred embodiments, other embodiments will become apparent to one skilled in the art from consideration of the specification. The invention is further defined by reference to the following examples describing in detail the pharmaceutical compositions and processes for preparing the pharmaceutical compositions of the invention. It will be apparent to those skilled in the art that many modifications, both to materials and methods, may be practiced without departing from the scope of the invention.

### EXAMPLES

### Example 1

| | | | | | | |
|---|---|---|---|---|---|---|
| | Formulation | Formulation | Formulation | Formulation | Formulation | Formulation |
| Raw Material | A (mg/Tab) | B (mg/Tab) | C (mg/Tab) | D (mg/Tab) | E (mg/Tab) | F (mg/Tab) |

Part I

| | | | | | | |
|---|---|---|---|---|---|---|
| Candesartan Cilexetil | 32.0 | 32.0 | | | 32.0 | |
| Lactose Mono. 200 | 121.7 | 184.1 | 38.0 | 38.0 | 185.7 | 38.0 |
| Mesh | | | | | | |
| Starch | 40.0 | 34.8 | | | 34.8 | |
| Povidone (PVP K-30) | 13 | | | | | |
| Starch 1500 | | | 50.0 | 50.0 | | 50.0 |
| Klucel lF | | 6.4 | | | 3.2 | |
| Carboxymethylcellulose | 5.2 | 6.4 | | | 6.4 | |
| Calcium | | | | | | |
| Col. Ferric Oxide | 0.3 | 0.3 | | | 0.3 | |
| Red | | | | | | |
| Hydrog. Castor Oil | | | | | 8.0 | |
| Powder | | | | | | |

Granulation Solution

| | | | | | | |
|---|---|---|---|---|---|---|
| Lutrol ® F127 | 5.2 | 6.4 | 9.6 | 6.4 | | 3.2 |
| (Poloxamer 407) | | | | | | |
| Klucel LF | | 6.4 | | | 6.4 | |
| Alcohol 95% | + | + | + | + | + | + |

Part II

| | | | | | | |
|---|---|---|---|---|---|---|
| Candesartan Cilexetil | | | 32.0 | 32.0 | | 32.0 |
| Lactose Spray Dried | | | 104.5 | 107.7 | | 171.0 |
| Povidone (PVP K-30) | | | 16.0 | 16.0 | | 16.0 |
| Granulate Part I | | | 97.6 | 94.4 | | 91.2 |
| Starlac | 40.0 | 40.0 | 60.0 | 60.0 | 40.0 | |
| Carboxymethylcellulose | | | 6.4 | 6.4 | | |
| Calcium | | | | | | |
| Col. Ferric Oxide Red | | | 0.3 | 0.3 | | |

Part III

| | | | | | | |
|---|---|---|---|---|---|---|
| Magnesium Stearate | 2.6 | 3.2 | 3.2 | 3.2 | 3.2 | 3.1 |
| TOTAL WEIGHT | 260.0 | 320.0 | 320.0 | 320.0 | 320.0 | 320.0 |

PILOT BIO STUDY - FAST (%)

| AUC | 149.0 | 160 | 108 | 99.99 | 87.58 | 94.7 |
|---|---|---|---|---|---|---|
| Cmax | 200.0 | 226 | 124 | 116.43 | 75.69 | 100.3 |
| 90% Cl AUC | 132-169 | 140-183 | 94.5-123 | 89.93-111.18 | 78.76-97.38 | 83.1-108 |
| 90% Cl Cmax | 186-216 | 184-277 | 101-153 | 98.99-136.95 | 64.35-89.02 | 84.8-118.6 |
| | Formulation | Formulation | Formulation | Formulation | Formulation | Formulation |
| | G (mg/Tab) | H (mg/Tab) | I (mg/Tab) | J (mg/Tab) | K (mg/Tab) | L (mg/Tab) |

Part I

| | | | |
|---|---|---|---|
| Candesartan Cilexetil | 32.0 | 32.0 | |
| Lactose Mono. 200 | 165.2 | 165.2 | 38.0 |
| Mesh | | | |
| Starch | 40.0 | 40.0 | |
| Povidone (PVP K-30) | | | |
| Primellose | 9.6 | 9.6 | |
| Starch 1500 | | | 50.0 |
| Klucel lF | | | |
| Carboxymethylcellulose | | | |
| Calcium | | | |
| Col. Ferric Oxide Red | | | |
| Hydrog. Castor Oil | 12.8 | 12.8 | |
| Powder | | | |

Granulation Solution

| | | | |
|---|---|---|---|
| Lutrol ® F127 | | 0.8 | 6.4 |
| (Poloxamer 407) | | | |
| Klucel LF | | | |
| Plasdone S-360 | 9 | 9 | |
| Alcohol 95% | + | + | + |

Part II

| | | | | | | |
|---|---|---|---|---|---|---|
| Candesartan Cilexetil | | | 32.0 | 32.0 | 32.0 | 32.0 |
| | | | | | (22μ) | |
| Lactose Spray Dried | | | 107.7 | | | |
| Povidone (PVP K-30) | | | 16.0 | 32.0 | 32.0 | |
| Granulate Part I | | | 94.4 | | | |
| Starlac | | | 60.0 | | | |
| Carboxymethylcellulose | | | 6.4 | | | |
| Calcium | | | | | | |
| Col. Ferric Oxide Red | | | 0.3 | 0.3 | 0.3 | 0.3 |
| Primellose | 6.4 | 6.4 | | 16.0 | 16.0 | 6.4 |
| Pharmatose DCL14 | 41.8 | 41.0 | | 171.7 | 171.7 | 180.0 |
| Avicel PH 102 | | | | | | 22.4 |
| Starch 1500 | | | | 34.0 | 34.0 | 34.0 |
| L-Leucine | | | | 32.0 | 32.0 | 32.0 |
| HPC-L | | | | | | 9.6 |
| S.LS | | | | | | 1.6 |

Part III

| | | | | | | |
|---|---|---|---|---|---|---|
| Magnesium Stearate | 3.2 | 3.2 | 3.2 | 2.0 | 2.0 | 1.7 |
| TOTAL WEIGHT | 320.0 | 320.0 | 320.0 | 320.0 | 320.0 | 320.0 |

PILOT BIO STUDY - FAST (%)

| | | | | | | |
|---|---|---|---|---|---|---|
| AUC | 118 | 152 | 106 | 84 | 65 | 67 |
| Cmax | 123 | 175 | 118 | 69 | 55 | 51 |
| 90% Cl AUC | 98-142 | 126-188 | 94-119 | 72-99 | 55-77 | 57-79 |
| 90% Cl Cmax | 89-169 | 127-242 | 100-139 | 53-91 | 42-73 | 39-58 |

| | | |
|---|---|---|
| | Formulation | Formulation |
| Raw Material | M (mg/Tab) | N (mg/Tab) |

Part I

Candesartan Cilexetil
Lactose Mono. 200
Mesh
Starch
Povidone (PVP K-30)
Primellose
Starch 1500
Klucel lF
Carboxymethylcellulose
Calcium
Col. Ferric Oxide
Red
Hydrog. Castor Oil
Powder

Granulation Solution

Lutrol ® F127
(Poloxamer 407)
Klucel LF
Plasdone S-360
Alcohol 95%

Part II

| | | |
|---|---|---|
| Candesartan Cilexetil | 32.0 | 32.0 |
| Lactose Spray Dried | | |
| Povidone (PVP K-30) | 20.0 | 31.0 |
| Granulate Part I | | |
| Starlac | | |
| Carboxymethylcellulose | | |
| Calcium | | |
| Col. Ferric Oxide Red | 0.3 | 0.5 |
| Primellose | 11.2 | 17.5 |
| Pharmatose DCL14 | 131.5 | 200.0 |
| Avicel PH 102 | | |
| Starch 1500 | 50.0 | 160.0 |
| L-Leucine | 75 | 59 |
| HPC-L | | |
| S.LS | | |

Part III

| | | |
|---|---|---|
| Magnesium Stearate | | |
| TOTAL WEIGHT | 320.0 | 500.0 |

PILOT BIO STUDY - FAST (%)

| | | |
|---|---|---|
| AUC | 75 | 70 |
| Cmax | 59 | 60 |
| 90% Cl AUC | 65-85 | 61-80 |
| 90% Cl Cmax | 50-70 | 50-71 |

All bio-studies were run with the innovator tablet as a control.
All values in tables are mg/tablet unless otherwise stated.
Formulations J, K, L, M, and N are comparative examples.

### Method: Wet Granulate Process (Formulations A, B, E, G, and H):

Part I:

1) Sieve the Carboxym Calcium & Col. Ferric Oxide Red through Sieve # 80 mesh. 2) Load into High Shear Mixer all components of part I and mix.

Granulation Solution:

3) Dissolve the Lutrol® F127 in Alcohol 95% by mixing with stirrer to clear solution.

4) Add the solution from step 3 to High Shear Mixer from step 2 and mix.

5) Dry the wet granulate from step 4 in a fluid bed dryer.

6) Mill the dry granulate from step 5 through a Quadro Comil.

7) Transfer the milled granulate from step 6 to Y-Cone or Flow-Bin.

Part II:

8) Add the Starlac to Y-Cone or Flow-Bin from step 7 and mix.

Part III:

9) Screen the Mg. Stearate through # 50 mesh Sieve.

10) Add the Mg. Stearate from step 9 to Y-Cone or Flow-Bin from step 8 and mix.

11) Compress the final blend from step 10 to form tablets.

### Method: Direct Compression Process (Formulations C. D, F, I, J, K, L, M, and N):

Part I:

1) Load into High Shear Mixer all components of part I and mix.

Granulation Solution:

2) Dissolve the Lutrol® F127 in Alcohol 95% by mixing with stirrer to clear solution.

3) Add the solution from step 2 to High Shear Mixer from step 1 and mix.

4) Dry the wet granulate from step 3 in a fluid bed dryer.

5) Mill the dry granulate from step 4 through a Quadro Comil.

Part II:

6) Load into Y-Cone or Flow-Bin all components of part II and mix.

7) Sieve the blend from step 6 through Quadro Comil.

8) Transfer the sieved from step 7 to Y-Cone or Flow-Bin and mix.

Part III:

9) Screen the Mg. Stearate through # 50 mesh Sieve. 10) Add the Mg. Stearate from step 9 to Y-Cone or Flow-Bin from step 8 and mix. 11) Compress the final blend from step 10 to form tablets.

If any of Parts I, II, or II are not present, the process steps relating to that Part are not carried out, e.g. if none of the components of Part I are present, just Parts II and III are mixed together.

We claim:

1. A pharmaceutical composition comprising candesartan, a prodrug thereof, an analog thereof, or a derivative thereof and at least one non-ionic surfactant, wherein the non-ionic surfactant is present in about 0.01% to about 10% of the total weight of the composition.

2. The pharmaceutical composition of claim 1, wherein the non-ionic surfactant is present in about 0.01% to about 8% of the total weight of the composition.

3. The pharmaceutical composition of claim 1, wherein the non-ionic surfactant is present in about 0.01% to about 5% of the total weight of the composition.

4. The pharmaceutical composition of claim 1, wherein the prodrug, the analog or the derivative of candesartan is candesartan cilexetil.

5. The pharmaceutical composition of claim 1, wherein the non-ionic surfactant is selected from the group consisting of block-copolymers of polyoxypropylene (poly(propylene oxide)) and polyoxyethylene (poly(ethylene oxide)) and hydrogenated triglyceride esters of ricinoleic, oleic, and linoleic acids or combinations thereof.

6. The pharmaceutical composition of claim 1, wherein the non-ionic surfactant is a poloxamer, hydrogenated castor oil powder, or a combination thereof.

7. The pharmaceutical composition of claim 1, wherein the non-ionic surfactant is poloxamer 407.

8. The pharmaceutical composition of claim 1, further comprising one or more pharmaceutically acceptable excipients.

9. The pharmaceutical composition of claim 1, wherein the pharmaceutical composition is in the shape of a granule.

10. The pharmaceutical composition of claim 9, wherein the granules are compressed into a tablet.

11. A process for preparing a pharmaceutical composition comprising combining a prodrug thereof, an analog thereof, or a derivative thereof and at least one non-ionic surfactant, wherein the non-ionic surfactant is present in about 0.01% to about 10% of the total weight of the composition.

12. The process of claim 11, wherein the combining step comprises wet or dry granulating candesartan, a prodrug thereof, an analog thereof, or a derivative thereof and the non-ionic surfactant.

13. The process of claim 11, wherein the pharmaceutical composition further comprises one or more pharmaceutical acceptable excipients.

14. The process of claim 12, wherein the granulating step is a wet-granulating step.

15. The process of claim 11, wherein the non-ionic surfactant is present in about 0.01% to about 8% of the total weight of the composition.

16. The process of claim 11, wherein the non-ionic surfactant is present in about 0.01% to about 5% of the total weight of the composition.

17. The process of claim 11, wherein the prodrug, the analog or the derivative of candesartan is candesartan cilexetil.

18. The process of claim 11, further comprising compressing the pharmaceutical composition into a solid dosage form.

19. The process of claim 18, wherein the solid dosage form is a tablet.

20. A granule comprising candesartan, a prodrug thereof, an analog thereof, or a derivative thereof and at least one non-ionic surfactant, wherein the non-ionic surfactant is present in about 0.01% to about 10% by weight of the granule.

21. The granule of claim 20, wherein the granule is wet-granulated.

22. The granule of claim 20, further comprising one or more pharmaceutical acceptable excipients.

23. The granule of claim 20, wherein the non-ionic surfactant is present in about 0.01% to about 8% by weight of the granule.

24. The granule of claim 20, wherein the non-ionic surfactant is present in about 0.01% to about 5% of the granule.

25. The granule of claim 20, wherein the prodrug, the analog, or the derivative of candesartan is candesartan cilexetil.

26. A method of treating a patient suffering from a circulatory system disease comprising administering to a patient in need thereof a pharmaceutical composition having a therapeutically effective amount of candesartan, a prodrug thereof, an analog thereof, or a derivative thereof and at least one non-ionic surfactant, wherein the non-ionic surfactant is present in about 0.01% to about 10% of the total weight of the composition.

27. The method of claim 26, wherein the circulatory system disease is hypertension.

28. A pharmaceutical composition comprising candesartan, a prodrug thereof, an analog thereof, or a derivative thereof and at least one non-ionic surfactant, wherein the non-ionic surfactant is present in about 0.01% to about 10% of the total weight of the composition and the composition has a bioavailability measured as area under the curve (AUC) of about 149 to 160.

29. A method of preparing a pharmaceutical composition comprising candesartan, a prodrug thereof, an analog thereof, or a derivative thereof having a target relative AUC comprising:
a) providing a pharmaceutical composition comprising candesartan;
b) determining the relative AUC;
c1) if the relative AUC is less than the target relative AUC, then increasing the amount of non-ionic surfactant in the composition;
c2) if the relative AUC is more than the target relative AUC, then decreasing the amount of non-ionic surfactant in the composition; and
d) repeating the process starting with step (b) until the target relative AUC is obtained.

30. A pharmaceutical composition comprising as an active ingredient candesartan or candesartan cilexetil, the composition exhibiting a relative bioavailability, measured as area under the curve (AUC), of more than 1.5.

31. The pharmaceutical composition of claim 31, wherein the composition further comprises a non-ionic surfactant.
